(19) United States
(12) Patent Application Publication
(10) Pub. No.: US 2023/0211149 A1
(43) Pub. Date: Jul. 6, 2023

(54) DEVICE AND METHOD FOR TREATING SINUS DISCOMFORT

(71) Applicant: TIVIC HEALTH SYSTEMS INC., Menlo Park, CA (US)

(72) Inventors: Blake Taylor Gurfein, OAKLAND, CA (US); Christopher A. Wiklof, Everett, WA (US)

(21) Appl. No.: 18/073,449

(22) Filed: Dec. 1, 2022

Related U.S. Application Data
(63) Continuation-in-part of application No. PCT/US21/34926, filed on May 28, 2021.
(60) Provisional application No. 63/032,811, filed on Jun. 1, 2020.

(51) Int. Cl. A61N 1/04

(52) U.S. Cl. CPC A61N 1/0456; A61N 1/0472

(57) ABSTRACT

In an embodiment, a sinus treatment apparatus includes a current generation circuit and first and second electrodes operatively coupled to the current generation circuit. The first electrode is configured to apply electric current to a treatment location on a skin surface of the user superjacent to a targeted anatomical structure coupled to the user's sinus. The second electrode is configured to provide a current return path to the current generation circuit from a second location on the surface of the user medial to the user's hand. In an embodiment, a method of using the sinus treatment apparatus includes receiving a return current through the user's body from a location other than the user's palm.

### CROSS-REFERENCE TO RELATED APPLICATIONS

The present application is a Continuation-in-Part application which claims priority benefit of co-pending International Patent Application No. PCT/US21/34926, entitled “DEVICE FOR TREATING SINUS TARGETS,” filed May 28, 2021 (docket number 3048-041-04), which claims priority benefit from U.S. Provisional Patent Application No. 63/032,811, entitled “DEVICE FOR TREATING SINUS TARGETS,” filed Jun. 1, 2020 (docket number 3048-041-02).

The present application is related to co-pending U.S. Non-Provisional application Ser. No. 17/516,452, entitled “SINUS TREATMENT DEVICE WITH ADAPTIVE CIRCUIT”, filed Nov. 1, 2021 (docket number 3048-001-07).

The present application is related to co-pending U.S. Non-Provisional application Ser. No. 16/664,085, entitled “ADAPTIVE TRIGGER FOR A MICROCURRENT STIMULATION DEVICE”, filed Oct. 25, 2019 (docket number 3048-031-05).

Each of the above applications, to the extent not inconsistent with the disclosure herein, is incorporated by reference.

### SUMMARY

According to an embodiment, a sinus treatment apparatus includes an electric current generation circuit having two output nodes, configured to output therapeutic electric current between the two nodes. A first electrode may be operatively coupled to the first node of the electric current generation circuit, configured to couple electrical current between the first node of the electric current generation circuit and a first selected location on a skin surface of a user's body. A second electrode may be operatively coupled to the second node of the electric current generation circuit, configured to contact a second location on the skin surface of the user's body different than the first location and to form a current path between the second location and the second node of the electric current generation circuit. The electric current generation circuit may be disposed inside a hand-holdable housing, and the first electrode may protrude through the hand-holdable housing. The first and the second electrodes may contact the first and the second locations on the skin surface of the user's body selected to cause a current path to form along a targeted anatomical structure, such as a nerve fiber, subcutaneous to the first location. The second location may lie medial to the user's hand.

According to an embodiment, a method for sinus treatment includes operating a sinus treatment apparatus having two modes. In a detection mode of the sinus treatment apparatus, the method includes detecting with a first electrode of the sinus treatment apparatus, a treatment location on a skin surface of a user's body. The treatment location corresponds to a nerve beneath the skin surface. When the treatment location is detected, the method includes transitioning from the detection mode to a treatment mode of the sinus treatment apparatus. The treatment mode includes providing an indication to the user to maintain contact of the first electrode with the detected treatment location while an electrical stimulation including treatment current is applied through a portion of the user's body.

### BRIEF DESCRIPTION OF THE DRAWINGS

FIG. 1 is a diagram of a sinus treatment apparatus for applying current to nerves of a human face, according to an embodiment.

FIG. 2 is an illustration of a face of a user of the sinus treatment apparatus highlighting treatment areas, according to an embodiment.

FIG. 3 is a view of the sinus treatment apparatus, according to an embodiment.

FIG. 4 is a view of the sinus treatment apparatus, according to another embodiment.

FIG. 5 is a flowchart for a method of operating a sinus treatment apparatus, according to an embodiment.

### DETAILED DESCRIPTION

In the following detailed description, reference is made to the accompanying drawings, which form a part hereof. In the drawings, similar symbols typically identify similar components, unless context dictates otherwise. Other embodiments may be used and/or other changes may be made without departing from the spirit or scope of the disclosure.

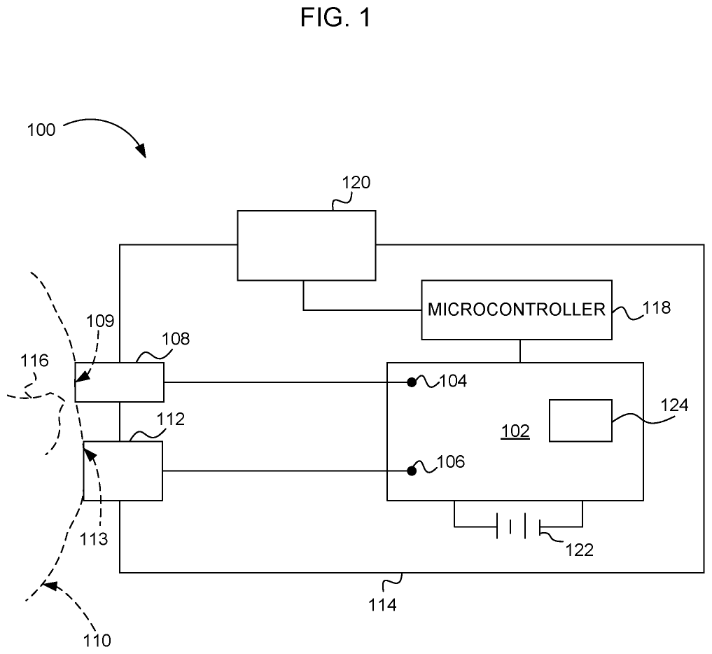

FIG. 1 is a diagram of a sinus treatment apparatus 100 for applying current to nerves of a human face, according to an embodiment.

According to an embodiment, a sinus treatment apparatus 100 includes an electric current generation circuit 102 having two output nodes 104, 106, configured to output therapeutic electric current between the two nodes 104, 106. The sinus treatment apparatus 100 may include a first electrode 108 operatively coupled to the first node 104 of the electric current generation circuit 102, configured to couple electrical current between the first node of the electric current generation circuit and a first selected location 109 on a skin surface 110 of a user's body (e.g., face). For example, the first electrode 108 may be held against the first selected location 109 without adhering to the first selected location 109. The sinus treatment apparatus 100 may include a second electrode 112 operatively coupled to the second node 106 of the electric current generation circuit 102, configured to contact a second location 113 on the skin surface 110 of the user's body different than the first location 109 and to form a current path between the second location 113 and the second node 106 of the electric current generation circuit 102. The second electrode 112 may be held in contact with the second location 113 using various approaches such as gravity, an elastic structure, a mechanical extension, or others, without being held in contact by an adhesive. The electric current generation circuit 102 may be disposed inside a hand-holdable housing 114, and the first electrode 108 may protrude through the hand-holdable housing 114. The first and the second electrodes 108, 112 may contact the first and the second locations 109, 113 on the skin surface 110 of the user's body selected to cause a current path to form along a targeted anatomical structure 116 subcutaneous to the first location 109. For example, the targeted anatomical structure 116 may include a nerve fiber or vascular structure.

As used herein, the terms nerve fiber and nerve node shall be considered synonymous unless context indicates otherwise. The term subcutaneous will be understood to refer to a location of a targeted anatomical structure, including nerve fiber or node or vascular structure lying beneath an outermost surface of the human body, whether the outermost surface comprises skin or mucous membrane. The second location 113 may lie medial to a grip surface of the user's hand. The term medial to a grip surface of the user's hand shall be understood to include any location on the surface of the user's body that does not include the palmar, or ventral, surfaces of the user's gripping hand, e.g., the palm and the ventral/medial surfaces of the thumb, or fingers or other hand parts that would contact the hand-holdable housing 114.

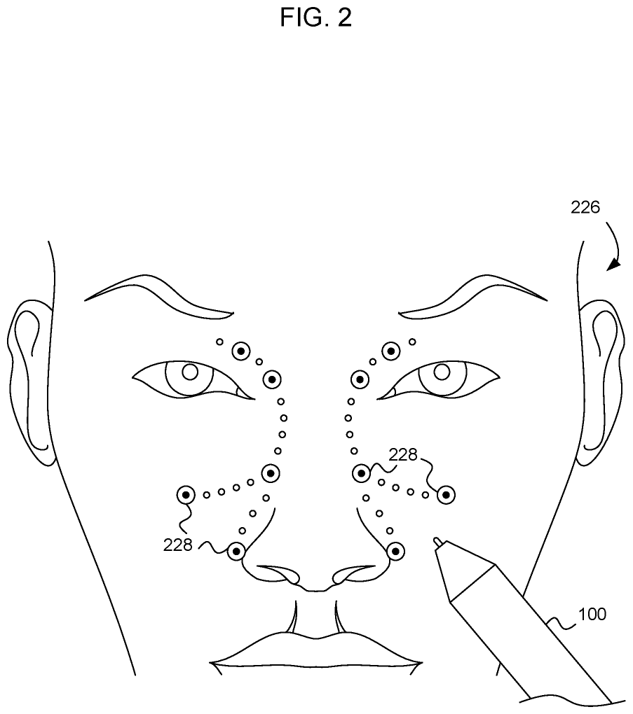

FIG. 2 illustrates locations of nerve nodes 228 on a human face 226 to which current may be applied and that have proven to be effective for treatment of sinus pain symptoms. According to an embodiment, treatment areas correspond to nerve nodes 228. The nerve nodes 228 are treatment locations at which sinus nerves provide low impedance electrical transmission paths. As used herein, the terms nerve nodes, treatment locations, treatment regions and treatment areas shall be considered synonymous unless further definition is provided.

According to an embodiment, a user uses the sinus treatment apparatus 100 by holding the hand-holdable housing of the sinus treatment apparatus 100 in one hand. The user places the first electrode on a surface adjacent to the sinuses while the second electrode contacts a location on the skin different than the location of the first electrode, the second electrode lying medial to a gripping surface of the one hand. The user glides the first electrode over the surface during a detection mode of the sinus treatment apparatus 100. In the detection mode, the sinus treatment apparatus 100 detects the treatment location, corresponding to the location of a nerve node 228 beneath the surface. When the sinus treatment apparatus 100 detects the treatment location of a nerve node 228 beneath the surface, the sinus treatment apparatus 100 can enter a treatment mode.

In one embodiment, the sinus treatment apparatus 100 detects nerve nodes 228 by detecting an impedance between the first electrode and the second electrode. Nerve nodes 228 are characterized by a lower impedance than surrounding areas due to enhanced conductivity of nerves.

According to an embodiment, in the treatment mode, the sinus treatment apparatus 100 provides treatment stimulation to the nerve node 228, corresponding to the nerve that is located during the detection mode. The electrical stimulation can affect the nerve node 228 in such a way that the user experiences relief from troubling sinus symptoms such as pain, congestion, inflammation, or other unpleasant symptoms.

According to an embodiment, the stimulation current is a DC current. In another embodiment, the stimulation current is characterized by current spikes. In an embodiment, the current spikes have an alternating polarity. According to an embodiment, the treatment stimulation is provided at each nerve node 228 for a period of time between 4-20 seconds.

According to an embodiment, the sinus treatment apparatus 100 applies the stimulation current by applying a stimulation voltage between the first electrode and the second electrode.

According to an embodiment, the first electrode is the active electrode of a monopolar design. A user's body location contacting the second electrode of the sinus treatment apparatus 100 completes the electrical path from the first electrode to the second electrode(s) in that currents may travel between the first electrode, through an underlying nerve node 228 of a user and back to a location, medial to the gripping surface of the user's hand, that is contacting the second electrode(s), in an embodiment. These currents may be referred to as “stimulation currents” in this disclosure.

According to an embodiment, in the detection mode, the user presses the first electrode to the skin and the sinus treatment apparatus 100 initiates a circuit that is maintained at a constant current. The sinus treatment apparatus 100 may use the current to calculate the impedance in the path in the tissue between the first location contacting the first electrode and the second location in contact with the second electrode. The sinus treatment apparatus 100 remains in the detection mode until the detection current indicates that the impedance is below a threshold impedance. The position of the first electrode when the impedance is below the threshold impedance corresponds to a treatment area 228. The treatment area 228 corresponds to a nerve node 228 area. When the sinus treatment apparatus 100 identifies a nerve node 228 based on the calculated impedance, the sinus treatment apparatus 100 can enter the treatment mode and can deliver treatment stimulation to the identified nerve node 228.

According to an embodiment, the sinus treatment apparatus 100 can indicate to the user that the sinus treatment apparatus 100 is in the treatment mode and that the user should hold the first electrode at the first location corresponding to the nerve node 228 for a selected period of time. According to an embodiment, the sinus treatment apparatus 100 can indicate the transition between the detection mode and the treatment mode by indicators. The indicators can include one or more LEDs that can provide an illumination scheme that indicates whether the sinus treatment apparatus 100 is in the detection mode or the treatment mode. According to an embodiment, the sinus treatment apparatus 100 can indicate that the sinus treatment apparatus 100 is in the treatment mode via haptic feedback (vibration). According to an embodiment, the sinus treatment apparatus 100 can indicate whether the sinus treatment apparatus 100 is in the detection mode, the treatment mode, or transitioning between the detection and treatment nodes by a combination of haptic feedback and LED indicators. According to an embodiment, when the sinus treatment apparatus 100 enters the treatment mode as indicated by one or more of LED indicators and haptic feedback, the user holds the sinus treatment apparatus 100 in place until the treatment period has passed as indicated by cessation of haptic and LED indicators (approximately 8 seconds in one example).

According to an embodiment, once the treatment period ends, the sinus treatment apparatus 100 resets to detection mode. The user then may continue to glide the first electrode along the indicated path until reaching the next nerve node 228 as identified based on impedance calculations. The user may adjust the impedance sensitivity of the sinus treatment apparatus 100, in one embodiment. Changes in sensitivity adjust the impedance threshold at which the sinus treatment apparatus 100 will enter treatment mode. Changes in sensitivity do not change the output current, in one embodiment.

In one embodiment of a treatment circuit of the disclosed sinus treatment apparatus 100, the constant current stimulation output is approximately 1 Hz-1000 Hz, bi-phasic, no DC component signal with an average current—less than 1000 μA over a resistive load of 10K-100KΩ. The signal is presented to the patient by means of the conductive tip, in one embodiment.

According to an embodiment, constant current stimulation circuit output is directed to the first electrode and returned to the circuit by way of the second electrode. When the circuit is completed by the user pressing the device first electrode to the face 226, a microcontroller (see, e.g., microcontroller 118 in FIG. 1) monitors the resulting stimulation current and controls the stimulation voltage (across the first electrode and the second electrode) to maintain the desired current, in one embodiment. The impedance of the circuit is then calculated and monitored by the microcontroller.

In one embodiment, the first and/or the second electrode includes an elastomeric material supporting a conductor, the elastomeric material being intended to minimize point pressure against the face 226 of the user. Various elastomers including silicone, fluorine-substituted silicones, natural rubber, vulcanized rubber, latex, latex derivatives, etc. may be used alone or in combination to form a support structure of the first electrode. In another embodiment, a non-elastomeric dielectric material such as a polymer, polymer combination, or glass may be used alone or in combination to form the support structure of the first and/or the second electrode. The support structure may be formed to have a relatively low thermal conductivity and/or may have a smooth radius to reduce point pressure against the skin of the user. Various conductive fibers or particles such as gold, silver, stainless steel, carbon fiber, carbon nanotubes, and/or alternating bond length (electron-conjugated) polymers are contemplated as current carriers supported by a dielectric support structure.

In one embodiment, the sinus treatment apparatus 100 includes a spring-loaded first electrode and/or second electrode. The first electrode may have a small surface area metalized feature treatment regions 228 of the face 226. In one embodiment, a microswitch initiates the therapy circuit when the first electrode is depressed. The sinus treatment apparatus 100 may include a microprocessor microcontroller, a battery, and a transformer/voltage step-up circuit. In one embodiment, the second electrode is a large surface area that is in contact with the user at a location medial to a gripping surface of the user's hand holding the sinus treatment apparatus, as defined above.

In one embodiment, the user interface of the sinus treatment apparatus 100 includes an LED treatment indicator (e.g., LEDs), a sensitivity level adjustment button, and a haptic feedback circuit. The LED sensitivity level indicates selected sensitivity levels in addition to low battery and charge status, and on/off button with integrated LED(s) to indicate “on” or “off” state, and a haptic feedback circuit.

In one embodiment, the sinus treatment apparatus 100 includes a battery charging port and circuit to charge an internal battery.

The inventors believe that some effects related to the noted therapeutic benefits may relate to electric current excitation of the vagus nerve, with which the facial nodes communicate.

Referring again to FIG. 1, according to an embodiment, the electric current generation circuit 102 includes a filter or regulator to control current output. In another embodiment, the electric current generation circuit 102 includes a filter or regulator to control voltage output. Additionally and/or alternatively, the electric current generation circuit 102 includes an electric pulse generation circuit configured to output a series of therapeutic electric pulses. The series of therapeutic electric pulses may include pulses having alternating polarity. In one embodiment, the electric current generation circuit 102 is configured to output less than 300 microamperes (uA) average between the two nodes 104, 106. In an embodiment, the electric current generation circuit 102 is configured to output about 120 uA average current. In an embodiment, the electric current generation circuit 102 is configured to output less than 15 milliamperes (mA) peak between the two nodes 104, 106. In an embodiment, the electric current generation circuit 102 is configured to output about 6 mA peak.

According to an embodiment, the sinus treatment apparatus 100 further includes a microcontroller 118 operatively coupled to the electric current generation circuit 102. The microcontroller 118 may be configured to control operation of the electric current generation circuit 102.

According to an embodiment, the sinus treatment apparatus 100 may further include a user-accessible switch 120 operatively coupled to the microcontroller 118. The user-accessible switch 120 may be configured to cause the microcontroller 118 to enable operation by the electric current generation circuit 102.

According to an embodiment, the sinus treatment apparatus 100 may further include an electric storage cell or battery 122 disposed inside the hand-holdable housing 114 and operatively coupled to the electric current generation circuit 102. The electric current generation circuit 102 may be configured to increase voltage provided by the electric storage cell or battery 122 to a therapeutic current application voltage.

In one embodiment, the first and the second electrodes 108, 112 are spaced apart sufficiently to cause current conduction from the first electrode 108 through a targeted anatomical structure 116 to be equal to or greater than current conduction along the skin surface 110 of the human body. In another embodiment, the first and the second electrodes 108, 112 are spaced apart a distance greater than a depth of the targeted anatomical structure 116 below the skin surface 110 of the human body. Additionally and/or alternatively, the first and the second locations 109, 113 where the first and the second electrodes 108, 112 contact the skin surface 110 of the human body are spaced at least a half centimeter apart. In another embodiment, the first and second electrodes 108, 112 are arranged to contact the skin surface 110 at least an inch apart. In an embodiment, the first electrode 108 has a contact area less than half a contact area of the second electrode 112. Additionally or alternatively, the second electrode 112 may have a contact area greater than three times a contact area of the first electrode 108. For example, the second electrode may have a contact area with the user's skin greater than ten times the contact area of the first electrode with the user's skin.

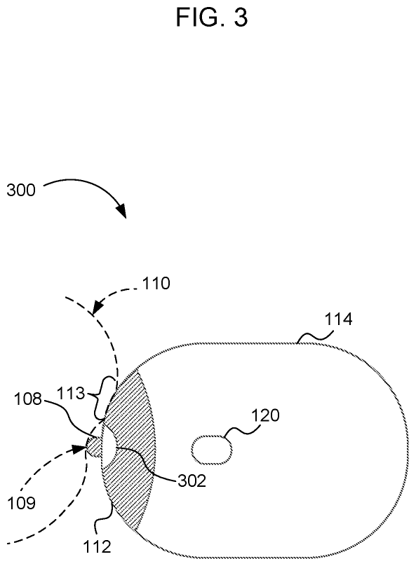

FIG. 3 is a view of a sinus treatment apparatus 300, according to an embodiment.

According to an embodiment, the second electrode 112 is formed on a surface of the hand-holdable housing 114. The hand-holdable housing 114 may be shaped to cause the second electrode 112 to contact the skin surface 110 of the user's face at the second location 113 when the first electrode 108 is pressed against the skin surface 110 of the user's face at the first location 109.

According to an embodiment, a sinus treatment apparatus 300 further includes a dielectric region 302 on the surface of the hand-holdable housing 114. The dielectric region 302 may be shaped to cause a separation distance between the first location 109 and the second location 113 on the skin surface 110 of the user's face. In one embodiment, a region of the hand-holdable housing 114 supporting the second electrode 112 includes an elastic member configured to cause the second electrode 112 to conform to the skin surface 110 of the user's face. The second electrode 112 may be disposed to be flexibly urged into contact with the skin surface 110 of the user's face at the second location 113 when the first electrode 108 is pressed into contact with the skin surface 110 of the user's face at the first location 109.

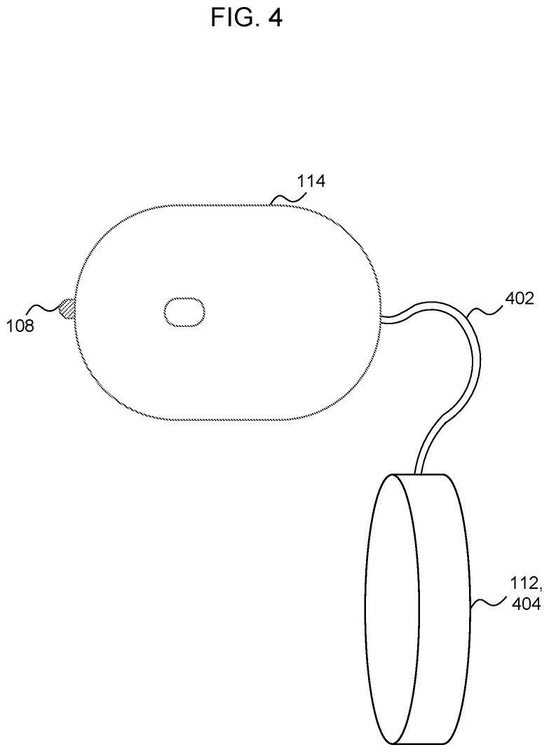

FIG. 4 is a view of a sinus treatment apparatus 400, according to an embodiment.

According to an embodiment, the sinus treatment apparatus 400 further includes a flexible coupling 402 between the hand-holdable case 114 and the second electrode 112. In one embodiment, the second electrode 112 includes a wrist strap 404 that makes electrical contact with the user's wrist. For example, the wrist strap 404 may include an elastic member that holds the wrist strap against the user's skin near the wrist. In another embodiment, the second electrode 112 is flexible. Additionally and/or alternatively, the second electrode 112 includes a flexible member configured to drape over the user's shoulder. For example, the second electrode 112 may include a weighted end configured to keep the second electrode 112 in contact with the user's shoulder under the influence of gravity.

In an embodiment, the first location 109 on the skin surface 110 of the user's body includes a location on the user's face. In another embodiment, the first location 109 on the skin surface 110 of the user's body includes one of several treatment locations at or adjacent to the user's nose. Additionally and/or alternatively, the first location 109 on the skin surface 110 of the user's body does not include a mucus membrane (e.g., inside a nostril).

Referring again to FIG. 1, according to an embodiment, the sinus treatment apparatus 100 further includes, operatively coupled to the electric current generation circuit 102, a sensing circuit 124 configured to detect a reduced impedance to current flow between the first and the second electrodes 108, 112. The electric current generation circuit 102 may be configured to be triggered to output the therapeutic current when the sensing circuit 124 detects that the first electrode 108 is at the first location 109 corresponding to a location of reduced impedance. In one embodiment, the first electrode 108 includes a radiused conductor extending from the hand-holdable housing 114. In another embodiment, the first and the second electrodes 108, 112 are disposed coaxially. Additionally and/or alternatively, the first and the second electrodes 108, 112 are arranged side-by-side. The second electrode 112 may have a larger contact patch 113 than the contact patch 109 of the first electrode 108. As used herein, the terms first location, second location, and contact patch shall be considered synonymous unless further definition is provided.

According to an embodiment, the sinus treatment apparatus 100 further includes a user-accessible switch 120, and a microcontroller circuit 118. The microcontroller circuit 118 may be configured to receive input from the user-accessible switch 120, enter a detection mode to detect alignment of the first electrode 108 with a treatment location (aka, first location) 109 for stimulating the targeted anatomical structure 116 associated with the sinuses of the user, and upon detecting the treatment location 109, enable the electric current generation circuit 102 to output a therapeutic current.

According to another embodiment, the sinus treatment apparatus 100 further includes a user-accessible switch 120, and a microcontroller circuit 118. The microcontroller circuit 118 may be configured to receive input from the user-accessible switch 120, enter a detection mode to detect alignment of the first electrode 108 with a treatment location 109 for stimulating a targeted anatomical structure 116 associated with the sinuses of the user, and upon detecting the treatment location 109 on the skin surface 110, enable an indicator to the user to maintain contact between the first electrode and the skin surface 110 at the first location while the electric current generation circuit 102 outputs a therapeutic current comprising a series of electric pulses.

In one embodiment, the microcontroller circuit 118 includes a microprocessor circuit. In another embodiment, the microcontroller circuit 118 includes a wireless interface (not shown).

According to an embodiment, the first and the second electrodes 108, 112 contact the first and the second locations 109, 113 on the skin surface 110 of the user's body selected to cause a current path to form along the targeted anatomical structure 116 such as a nerve fiber or vascular structure.

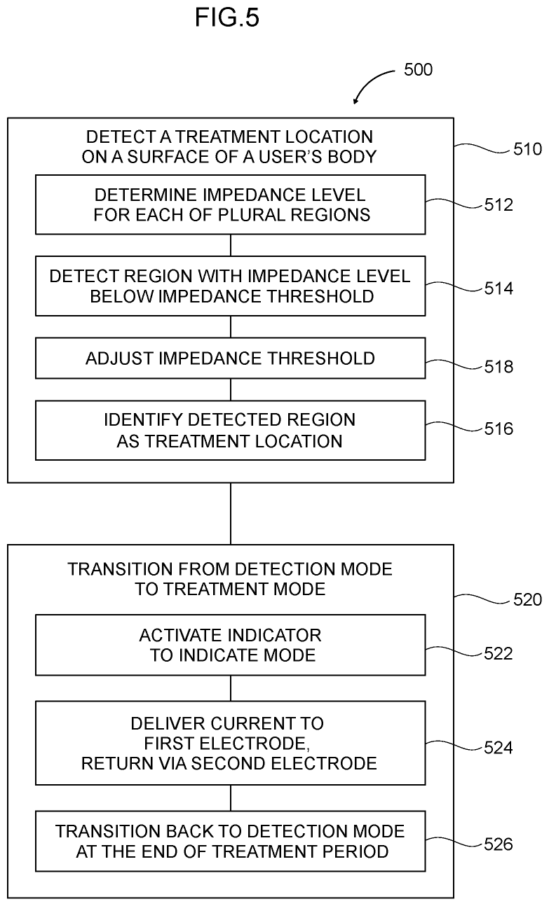

FIG. 5 is a flowchart for a method 500 for operating a sinus treatment apparatus (e.g., sinus treatment apparatus 100), according to an embodiment. The method 500 includes operating the sinus treatment apparatus in two modes: a detection mode and a treatment mode. In step 510, the method includes, in the detection mode, detecting, with a first electrode of the sinus treatment apparatus, a treatment location on a skin surface of a user's body. The treatment location may correspond to a nerve beneath the skin surface. In step 520, the method includes, when a treatment location is detected, transitioning from the detection mode to the treatment mode of the sinus treatment apparatus. The treatment mode includes providing an indication to the user to maintain contact of the first electrode with the detected treatment location while an electrical stimulation including treatment current is applied through a portion of the user's body. The sinus treatment apparatus a second electrode having a contact area at least three times a contact area of the first electrode, the second electrode contacting the user's body at a location other than a gripping surface of a hand holding the sinus treatment apparatus and separated from the treatment location by at least one inch across the user's skin.

Step 510, detecting the treatment location, may include in Step 512, determining, respectively for each of a plurality of regions of the skin surface of the user's body, an impedance level between the first electrode and the second electrode of the sinus treatment apparatus when the first electrode is in contact with a respective region among the plurality of regions and the second electrode is in contact with the location other than the gripping surface of the hand holding the sinus treatment apparatus. In Step 514, the method includes detecting, among the plurality of regions, a region having an impedance level that is below an impedance of previously measured regions. In Step 516, the method may include identifying the detected region as the treatment location.

According to an embodiment, the providing of the electrical stimulation in Step 520 may include (Step 524) delivering a bipolar electrical current to the first electrode and returning the bipolar electrical current via the second electrode. The bipolar electrical current may result from providing a stimulation voltage between the first electrode and the second electrode.

According to another embodiment, the providing of the electrical stimulation in Step 520 may include providing a plurality of constant peak current pulses via the first electrode and returning the constant peak current pulses via the second electrode. In some implementations, the current pulses may alternate in polarity.

The identifying of a detected region as the treatment location, in Step 516 may include, operating one or more indicators perceivable by the user. This operating of one or more indicators may include changing an illumination of one or more LEDs. For example, a series of LEDs may be lit in sequence corresponding to level of impedance. According to an embodiment, a fully lit series of LEDs may indicate that the surface region at the electrode satisfies the impedance threshold. According to another embodiment a single LED may be activated to indicate satisfaction of the impedance threshold (e.g., an impedance greater than the impedance threshold).

According to embodiment, the method 500 may include adjusting a sensitivity to adjust magnitude of the treatment current. The adjustment of sensitivity may include receiving input from an adjustment actuator (e.g., a button, wheel, slider, or the like) to increase or decrease the treatment current. Alternatively, in some embodiments the method 500 may include automatically adjusting the peak current to one of a range of constant peak currents

The transitioning from the detection mode to the treatment mode of the sinus treatment apparatus in Step 520 may include, Step 522, operating one or more indicators perceivable by the user to indicate the treatment mode. Such operating of the one or more indicators may include changing an illumination of one or more LEDs corresponding to at least one of the detection mode and the treatment mode. For example, an LED may be lit to indicate the treatment mode, or may change from a first color that indicates the detection mode to a second color that indicates the treatment mode. In another embodiment, each mode may activate a respective LED (one LED for detection mode, another LED for treatment mode). According to an embodiment, the operating of the one or more indicators may include providing a haptic feedback at one or more positions of a housing of the sinus treatment apparatus. The user may thus receive a physical indication of the transition from detection mode to treatment mode and vice versa. According to an embodiment, the operating of the one or more indicators may be engaged during the providing of the electrical stimulation to the detected treatment location. For example, an indicator (e.g., LED, sound, vibration) may be activated to notify the user that treatment of the region of the user's body being touched by the sinus stimulation apparatus is receiving treatment.

The method 500 may include, in Step 526, transitioning the sinus treatment apparatus back to the detection mode from the treatment mode at the end of a treatment period. In some embodiments, the treatment period may end after a preset time. In other embodiments, an end of the treatment period may result from detection of a sufficient change in the impedance of the treatment region. The transitioning back to the detection mode may include operating the one or more indicators to indicate exiting the treatment mode and re-entering the detection mode.

According to an embodiment, the providing of the electrical stimulation to the detected treatment location in Step 520 may include outputting a stimulation current from a constant current stimulation circuit through a first electrode. The stimulation current is returned to the constant current stimulation circuit via a second electrode while the first electrode is in contact with the treatment location and the second electrode is in non-adhesive contact with the user's skin at a location other than the treatment location. The provision of the stimulation current may include maintaining the stimulation current at a constant amperage by monitoring the stimulation current and, based on the monitored stimulation current, controlling a stimulation voltage across the first electrode and the second electrode. According to an embodiment, outputting of the stimulation current may include outputting a series of therapeutic electric pulses, which pulses may have alternating polarity.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. A sinus treatment apparatus, comprising:
an electric current generation circuit having two output nodes, configured to output therapeutic electric current between the two nodes;
a first electrode operatively coupled to the first node of the electric current generation circuit, configured to couple electrical current between the first node of the electric current generation circuit and a first selected location on a skin surface of a user's body; and
a second electrode operatively coupled to the second node of the electric current generation circuit, configured to contact a second location on the skin surface of the user's body different than the first location and to form a current path between the second location and the second node of the electric current generation circuit;
wherein the electric current generation circuit is disposed inside a hand-holdable housing and wherein the first electrode protrudes through the hand-holdable housing;
wherein the first and the second electrodes are arranged to contact the first and the second locations on the skin surface of the user's body selected to cause a current path to form along a targeted anatomical structure subcutaneous to the first location; and
wherein the second location lies medial to a gripping surface of the user's hand and separated from the first location by least one inch.

2-10. (canceled)

11. The sinus treatment apparatus of claim 1,
wherein the first and second electrodes are spaced apart sufficiently to cause current conduction from the first electrode through a targeted anatomical structure to be equal to or greater than current conduction along the skin surface of the human body.

12. The sinus treatment apparatus of claim 1, wherein the first and second electrodes are spaced apart a distance greater than a depth of the targeted anatomical structure below the skin surface of the human body.

13. (canceled)

14. The sinus treatment apparatus of claim 1, wherein the second electrode has a contact area greater than three times a contact area of the first electrode.

15. The sinus treatment apparatus of claim 1,
wherein the second electrode is formed on a surface of the hand-holdable housing; and
wherein the hand-holdable housing is shaped to cause the second electrode to contact the skin surface of the user's face at the second location when the first electrode is pressed against the skin surface of the user's face at the first location.

16. The sinus treatment apparatus of claim 15, further comprising a dielectric region on the surface of the hand-holdable housing, the dielectric region being shaped to cause a separation distance greater than one inch between the first location and the second location on the skin surface of the user's face.

17. The sinus treatment apparatus of claim 15, wherein a region of the hand-holdable housing supporting the second electrode includes an elastic member configured to cause the second electrode to conform to the skin surface of the user's face.

18. The sinus treatment apparatus of claim 17, wherein the second electrode is disposed to be flexibly urged into contact with the skin surface of the user's face at the second location when the first electrode is pressed into contact with the skin surface of the user's face at the first location.

19. The sinus treatment apparatus of claim 1, further comprising:
a flexible coupling between the hand-holdable case and the second electrode.

20. The sinus treatment apparatus of claim 19, wherein the second electrode comprises a wrist strap that makes electrical contact with the user's wrist.

21. The sinus treatment apparatus of claim 19, wherein the second electrode is flexible.

22. The sinus treatment apparatus of claim 19, wherein the second electrode comprises a flexible member configured to drape over the user's shoulder.

23. The sinus treatment apparatus of claim 1, wherein the first location on the skin surface of the user's body comprises a location on the user's face.

24. The sinus treatment apparatus of claim 1, wherein the first location on the skin surface of the user's body comprises one of several treatment locations at or adjacent to the user's nose.

25. The sinus treatment apparatus of claim 1, wherein the first location on the skin surface of the user's body does not include a mucus membrane.

26. (canceled)

27. The sinus treatment apparatus of claim 1, wherein the first electrode includes a radiused conductor extending from the hand-holdable housing.

28. The sinus treatment apparatus of claim 1, wherein the first and the second electrodes are disposed coaxially.

29. The sinus treatment apparatus of claim 1, wherein the first and the second electrodes are arranged side-by-side.

30. The sinus treatment apparatus of claim 1, wherein the second electrode has a larger contact patch than the contact patch of the first electrode.

31-34. (canceled)

35. The sinus treatment apparatus of claim 1, wherein the first and the second electrodes contact the first and the second locations on the skin surface of the user's body selected to cause a current path to form along a nerve fiber.

36. (canceled)

37. The sinus treatment apparatus of claim 1, wherein the first electrode is configured to couple electrical current to the first selected location without adhering to the first selected location; and
wherein the second electrode is configured to contact the second location without adhering to the second location, without being held in contact by an adhesive.

38-57. (canceled)
